(19) United States
(12) Patent Application Publication
(10) Pub. No.: US 2009/0264674 A1
(43) Pub. Date: Oct. 22, 2009

(54) SYSTEM AND METHOD FOR PRODUCING DIESTER

(75) Inventors: Hsiao-Ping HUANG, Taipei (TW); Cheng-Ching YU, Taipei (TW); Ming-Jer LEE, Taipei (TW); Shih-Bo HUNG, Taipei (TW); I-Kuan LAI, Taipei (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: NATIONAL TAIWAN UNIVERSITY, Taipei (TW)

(21) Appl. No.: 12/420,449

(22) Filed: Apr. 8, 2009

(30) Foreign Application Priority Data
Apr. 17, 2008 (TW) 097114029

(51) Int. Cl. C07C 67/08; B01J 19/00

(52) U.S. Cl. 560/204; 422/198

(57) ABSTRACT

The system and method for producing diester are provided. The system includes a first distillation unit reacting a dicarboxylic acid with an alcohol to produce a first gaseous mixture, a first cooling unit connected to the first distillation unit and cooling the first gaseous mixture into a first liquid mixture, a second distillation unit connected to the first cooling unit and separating the first liquid mixture into a second gaseous mixture and a second liquid mixture, and a liquid-liquid extraction unit connected to the second distillation unit and separating the second liquid mixture into an aqueous-rich mixture and an organic-rich mixture from which the diester is obtained.

### FIELD OF THE INVENTION

The invention relates to a system and method for producing diester, and more particularly to a system and method for producing diester of C₄ or higher dicarboxylic acid.

### BACKGROUND OF THE INVENTION

Diesters of C₄ or higher dicarboxylic acids are organic reagents with high boiling points, high stability and low toxicity, and can practically substitute volatile and toxic organic reagents with strong smell, such as ethylene glycol monoethyl ether acetate, ethylene Glycol monobutyl ether, propylene glycol ether esters, cyclohexanone, diacetone alcohol and methyl phenol etc. Among the above diesters, diesters of C₄-C₆ dicarboxylic acids, such as dimethyl succinate, dimethyl glutarate and dimethyl adipate are considered as reagents that are more potential environmentally protective and safe, such as coating materials, plasticizers, solvents and paint strippers, which are widely used in chemical industries manufacturing plastics, pharmaceuticals, inks, resins, and so on.

The conventional manufacturing process for the above diesters is generally divided into two independent procedures. One is a reaction procedure of reacting dicarboxylic acids with alcohols to produce esters and other products and residual materials without esters; the other one is a separation procedure of obtaining ester products from an ester-rich organic phase since an ester is water immiscible.

To manufacture a large amount of the above esters, the industries typically employ the manufacturing processes as disclosed in U.S. Pat. No. 4,271,315 and U.S. Pat. No. 4,365,080. For example, to put the above reaction and separation procedures into practice, the process of manufacturing dimethyl esters of C₄-C₆ dicarboxylic acids is implemented through at least following stages:

(1) Esterification (reaction): feed materials of dicarboxylic acids admixed with methanol in a stoichiometric ratio or the like are heated to carry out the esterification and form methyl esters of C₄-C₆ dicarboxylic acids.

(2) Extraction (separation): the methyl esters are extracted with a water immiscible organic solvent, in which an organic-rich and an aqueous-rich phases are separated.

(3) Recovery: the dimethyl ester products are obtained by further purifying and separating (e.g. rectifying) the organic-rich phase, and the methanol is recycled by stripping the aqueous-rich phase.

The aforementioned manufacturing process may obtain a large quantity of high-purity dimethyl esters of dicarboxylic acids, but it still has some problems to be solved. First, since the procedures of “reaction” and “separation” are independent from each other, many units must be involved therein which consumes a lot of time, materials, and cost. Moreover, the preliminary separation through the extraction must consume a large quantity of organic solvent and may require additional cost for processing the organic waste causing environmental pollution. Furthermore, the unesterified dicarboxylic acids in the esterification must be reacted again and recycled in the subsequent stage(s), so the manufacturing efficiency may not be desirable.

Therefore, to overcome the drawbacks from the prior art and to meet the present needs, the Applicant dedicated in considerable experiments and research, and finally accomplishes the “system and method for producing a diester” of the present invention.

The invention is briefly described as follows.

### SUMMARY OF THE INVENTION

To solve the problems of the prior art, the invention provides a system and method for producing a diester, especially for producing a diester of C₄ or higher dicarboxylic acid (preferably a diester of C₄-C₆ dicarboxylic acid). On basis of the differences among the boiling points of dicarboxylic acid, alcohol, and the reacted products thereof, the system and method of the invention are deigned to simultaneously carry out esterification and separation, which obtains diesters with at least industrial grade purity, and the application of the invention is simple, economical, environmental protective and time saving.

To achieve the above advantages, the system and method for producing a diester according to the invention mainly utilize distillation to simultaneously complete the reaction of dicarboxylic acid and alcohol and the preliminary separation of a mixture of alcohol, diester and water. Therefore, the involved units are less than those in the prior art, which could significantly save time, materials and cost. In addition, without the employment of an organic solvent in the preliminary separation, the pollution of a great quantity of organic waste can be avoided.

Furthermore, the system and method for producing a diester according to the invention can recycle the unreacted alcohol into the alcohol feed stock for reuse and adjust the ratio of fed dicarboxylic acid to fed alcohol over the stoichiometric ratio, so as to complete the reaction of the dicarboxylic and significantly increase the production efficiency.

In accordance with the first aspect of the present invention, a system for producing a diester is provided. The system includes a first distillation unit reacting a dicarboxylic acid with an alcohol to produce a first gaseous mixture, a first cooling unit connected to the first distillation unit and cooling the first gaseous mixture into a first liquid mixture, a second distillation unit connected to the first cooling unit and separating the first liquid mixture into a second gaseous mixture and a second liquid mixture, and a liquid-liquid extraction unit connected to the second distillation unit and separating the second liquid mixture into an aqueous-rich mixture and an organic-rich mixture from which the diester is obtained.

Preferably, the system further includes an ester recycle unit connected to the liquid-liquid extraction unit and separating the organic-rich mixture for obtaining the diester.

Preferably, the ester recycle unit is a third distillation unit.

Preferably, the dicarboxylic acid has a total carbon number of no less than 6.

Preferably, the dicarboxylic acid is one selected from a group consisting of succinic acid, glutaric acid, adipic acid and a combination thereof, and the alcohol is one selected from a group consisting of methanol, ethanol, isopropanol, butanol and a combination thereof.

Preferably, the dicarboxylic acid and the alcohol have a molar ratio ranged from 1.0:1.0 to 1.0:30.0.

Preferably, the first gaseous mixture is a mixture of the alcohol, the diester and water, the second gaseous mixture is an alcohol-rich mixture, and the second liquid mixture is a mixture of the diester and the water.

Preferably, the first distillation unit includes a plurality of reactive trays configured in a bottom to middle section of the first distillation unit, a plurality of rectifying trays configured in a middle to top section of the first distillation unit, and a solid catalyst filled in a middle to bottom section of the first distillation unit.

Preferably, the system further includes a first bypass device including a first end connected to the first distillation unit for receiving the first liquid mixture, a second end connected to the second distillation unit for introducing a first portion of the first liquid mixture into the second distillation unit for separating the first portion to produce the second gaseous mixture and the second liquid mixture, and a third end connected to the first distillation unit for returning a second portion of the first liquid mixture to the first distillation unit.

Preferably, the system further includes an alcohol feedstock connected to the first distillation unit and providing the alcohol, a second cooling unit connected to the second distillation unit and cooling the second gaseous mixture into a third liquid mixture, and an alcohol recycle path configured between the second distillation unit and the alcohol feedstock and recycling the third liquid mixture to the alcohol feedstock.

Preferably, the system further includes a second bypass device connected to the second distillation unit for splitting the third liquid mixture into a first portion to be introduced to the alcohol feedstock and a second portion to be returned to the second distillation unit.

Preferably, the second distillation unit has a single manipulated variable for controlling a reboiler duty and adjusting production of the second gaseous mixture. Preferably, the second distillation unit is a stripper. Preferably, the dicarboxylic acid is adipic acid.

Preferably, the second distillation unit has a first manipulated variable for controlling a reboiler duty and adjusting production of the second gaseous mixture and a second manipulated variable for controlling a reflux ratio and adjusting a proportion of the diester in the third liquid mixture. Preferably, the second distillation unit is a typical distillation column. Preferably, the dicarboxylic acid is glutaric acid.

In accordance with the second aspect of the present invention, a method for producing a diester is provided. The method includes the steps of admixing a dicarboxylic acid and an alcohol to produce a first gaseous mixture via distillation, cooling the first gaseous mixture to form a first liquid mixture, separating the first liquid mixture by the distillation to produce a second gaseous mixture and a second liquid mixture, separating the second liquid mixture into an organic-rich mixture and an aqueous-rich mixture by liquid-liquid extraction, and separating the organic-rich mixture to produce the diester.

Preferably, the method further includes a step of cooling the second gaseous mixture to form a third liquid mixture and introducing the third liquid mixture into the alcohol.

Preferably, the organic-rich mixture is separated by distillation to produce the diester.

The objects and advantages of the present invention will become more readily apparent to those ordinarily skilled in the art after reviewing the following descriptions with reference to the accompanying drawings, in which:

### BRIEF DESCRIPTION OF THE DRAWINGS

FIG. 1 represents a diagram illustrating the system for producing a diester according a preferred embodiment of the invention.

FIG. 2 represents a diagram illustrating the system for producing a diester according to a first varied embodiment of FIG. 1.

FIG. 3 represents a diagram illustrating the system for producing a diester according to a second varied embodiment of FIG. 1.

FIG. 4 represents a diagram illustrating the system for producing a diester according to a third varied embodiment of FIG. 1.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only, and it is not intended to be exhaustive or to be limited to the precise form disclosed.

The system and method for producing a diester according to the present invention are mainly designed for improving the reactive distillation column for carrying our esterification. The esterification for producing the diester can be depicted by the general formula as follows.

$$\begin{matrix}dibasic acid + alcohol {⇔}_{{k}_{- 1}}^{{k}_{1}} monoester + water \\ monoester + alcohol {⇔}_{{k}_{- 2}}^{{k}_{2}} diester + water\end{matrix}$$

The above reactions are typically reversible. In the above equations, the dibasic acid refers to a C₄ or higher dicarboxylic acid, particularly succinic acid, glutaric acid, adipic acid or the combination thereof; the alcohol refers to methanol, ethanol, isopropanol or butanol, particularly butanol; the diester yielded from the above reactions refers to a C₄ or higher dicarboxylic acid, particularly dimethyl succinate, dimethyl glutarate, dimethyl adipate or the combination thereof.

According to the physicochemical property of the entire reaction system, a diester of C₄ or higher dicarboxylic acid and water have an azeotropic point. Since the azeotropic point is much lower than the boiling points of the dicarboxylic acid and the residual monoester, the mixture of the water and the diester and the alcohol with a lower boiling point would become gaseous via distillation and will be preliminarily separated therefrom.

For example, in order to produce dimethyl glutarate, the system is operated by reacting methanol with glutaric acid to yield dimethyl glutarate. In this reaction system, in addition to dimethyl glutarate, the product also contains water and the residual unreacted methanol, glutaric acid, and monomethyl glutarate produced from the incomplete reaction. The boiling points of the various components are listed in the following Table 1.

TABLE 1

| Component | Boiling point (° C.) | |
|---|---|---|
| Methanol | 64.53° | C. |
| Water/dimethyl glutarate | 98.28° | C. |
| Water | 100.0° | C. |
| Monomethyl glutarate | 172.28° | C. |
| Dimethyl glutarate | 197.12° | C. |
| Glutaric acid | 322.13° | C. |

Based on the above Table 1, the boiling points of methanol, the mixture of water and dimethyl glutarate, and water are much lower than monomethyl glutarate (produced from the incomplete reaction) and glutaric acid. Through distillation, the water and the dimethyl glutarate are turned to be a constant boiling mixture, and the mixture, the methanol and water become gaseous, so that the dimethyl glutarate and the glutaric acid can be preliminarily separated from each other. Therefore, the procedures of “esterification” and “separation of dimethyl glutarate” can be simultaneously performed in the same one unit. Moreover, the residual glutaric acid can continually react with the supplied methanol so that the glutaric acid can be fully used and continually yield the dimethyl glutarate.

The two components of ester and water have the azeotropic point that will form a distillation boundary, so that a single reactive distillation column is difficult to obtain a high-purity ester product. Therefore, the present invention further includes an additional separation procedure/unit for additional purification and separation to obtain a high-purity dimethyl glutarate product.

For example, in the system for producing dimethyl adipate, the boiling points of the various components are listed in the following Table 2.

TABLE 2

| Component | Boiling point (° C.) | |
|---|---|---|
| Methanol | 64.53° | C. |
| Water/dimethyl adipate | 99.77° | C. |
| Water | 100.0° | C. |
| Dimethyl adipate | 235.68° | C. |
| Monomethyl adipate | 261.67° | C. |
| Adipic acid | 337.47° | C. |

Similarly, through distillation, the procedures of “esterification” and “separation of dimethyl adipate” can be simultaneously carried out in the same one unit. Moreover, the residual adipic acid can continually react with the supplied methanol so that the adipic acid can be fully used and continually yield the dimethyl adipate.

Even if there is the same problem in the process of reaction distillation for producing dimethyl adipate and it is difficult to distill the product with the mid-boiling point, the high-purity dimethyl adipate product can be obtained by employing the additional separation procedure/unit for further purification and separation provided in the present invention.

For the purpose of manufacturing the above diesters, the method of the present invention is mainly carried out by admixing and reacting the fed dicarboxylic acid (such as succinic acid, glutaric acid, adipic acid or the combination thereof in any ratio) with the fed alcohol (such as methanol, ethanol, isopropanol or butanol) to yield the diester of the dicarboxylic acid (such as dimethyl succinate, dimethyl glutarate, dimethyl adipate or the combination thereof). A gaseous mixture of the alcohol, the diester and water separated through distillation is cooled and formed to be a liquid mixture of the alcohol, the diester and the water. As for the liquid mixture, an alcohol-rich gas is separated therefrom through the distillation, and a liquid mixture mainly of the diester and the water is remained. The alcohol-rich gas is cooled and turned into an alcohol-rich liquid to be recycled into the alcohol feedstock for reuse of the alcohol. The alcohol feed can be controlled, so that the molar ratio of the fed alcohol to the fed dicarboxylic acid exceeds the stoichiometric ratio so that above-mentioned reversible reactions turns to be forward reactions and elevate the manufacturing efficiency. For example, the molar ratio of the fed alcohol to the fed dicarboxylic acid is ranged from 1.0:1.0 to 1.0:30.0. Based on the principle that an organic phase is immiscible with aqueous phase, the liquid mixture mainly of the diester and the water are separated into an organic-rich mixture mainly of the diester and an aqueous-rich mixture. The organic-rich mixture is refined to yield the high-purity diester product.

For the purpose of manufacturing the above-mentioned diesters, the system of the present invention mainly includes a first distillation unit, a second distillation unit and a liquid-liquid extraction unit. The first distillation unit, e.g. reactive distillation column, receives and react a dicarboxylic acid (such as succinic acid, glutaric acid, adipic acid or the combination thereof in any ratio) and an alcohol (such as methanol, ethanol, isopropanol or butanol) and produce a diester of a dicarboxylic acid (such as dimethyl succinate, dimethyl glutarate, dimethyl adipate or the combination thereof) and separates a gaseous mixture mainly of the alcohol, the diester and water via distillation, in which the gaseous mixture flows through a first cooling unit e.g. a condensing tube connected to the first distillation unit and is cooled into a liquid mixture mainly of the alcohol, the diester and water.

The second distillation unit, e.g. stripper or conventional distillation column for recycling alcohol, is connected to the first distillation unit and an alcohol feedstock providing the alcohol, from which the liquid mixture mainly of the alcohol, the diester and the water is separated via distillation into an alcohol-rich gaseous mixture and a liquid mixture mainly of the diester and the water, in which the alcohol-rich gaseous mixture is to be cooled by e.g. a condensing tube into an alcohol-rich liquid mixture, and the alcohol-rich mixture is introduced into the alcohol feedstock for recycling and reusing the alcohol. The alcohol feed can be controlled, so that the molar ratio of the fed alcohol to the fed dicarboxylic acid exceeds the stoichiometric ratio so that above-mentioned reversible reactions turns to be forward reactions and elevate the manufacturing efficiency. For example, the molar ratio of the fed alcohol to the fed dicarboxylic acid is ranged from 1.0:1.0 to 1.0:30.0.

The liquid-liquid extraction unit, e.g. decanter, is connected to the second distillation unit or the first cooling unit. The liquid mixture mainly of the diester and the water is separated therefrom into an organic-rich mixture mainly of the diester and an aqueous mixture.

The system of the present invention may further include an ester recycle unit. The ester recycle unit, e.g. distillation column for recycling the ester, is connected to the liquid-liquid extraction unit, in which the organic-rich mixture is refined to be a high-purity diester product.

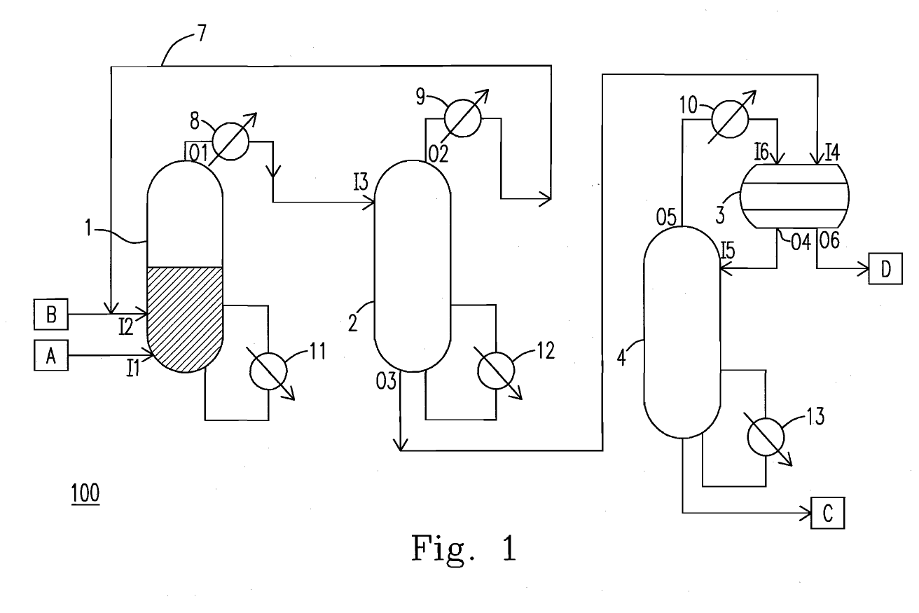

The practice of the system and method of the present invention can be better understood by reference to the diagrams of FIGS. 1 to 4 illustrating the system for producing a diester according preferred embodiments of the invention.

Referring to FIG. 1, it illustrates the system for producing a diester according a preferred embodiment of the invention. In FIG. 1, the basic system for producing a diester 100 mainly includes a first distillation unit 1, a second distillation unit 2, a liquid-liquid extraction unit 3 and an ester recycle unit 4. Preferably, there is an alcohol recycle path 7 configured between the second distillation unit 2 and an alcohol feedstock B. Preferably, the system is further configured with a first condenser 8 connected to the first distillation unit 1 and the second distillation unit 2, a second condenser 9 connected to the second distillation unit 2, and a third condenser 10 configured between the ester recycle unit 4 and the liquid-liquid extraction unit 3. Preferably, the system is further configured with a first reboiler 11, a second reboiler 12 and a third reboiler 13 respectively configured to the bottoms of the first distillation unit 1, the second distillation unit 2 and the ester recycle unit 4. To practice the method for producing the diester according to the invention, the operating pressure is ranged from 1 to 2 atm.

Based on the basic system for producing a diester 100, there are many varied embodiments for further increasing the efficiency of the system for producing a diester 100.

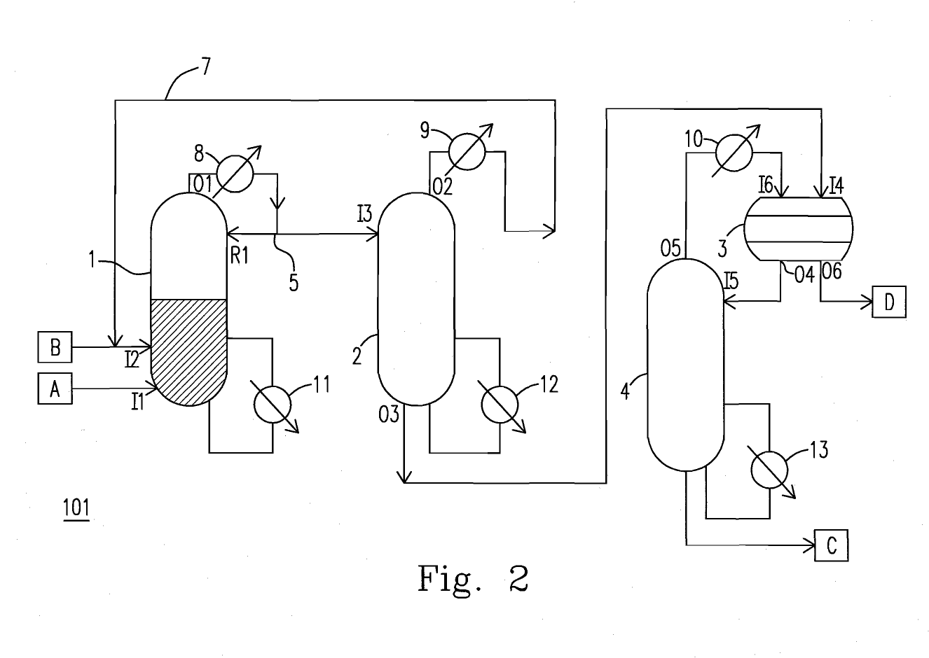

Referring to FIG. 2, it represents a diagram illustrating the system for producing a diester according to a first varied embodiment of FIG. 1. In FIG. 2, the system for producing a diester 101 according to a first varied embodiment is further configured with a first bypass device 5 connected between the first distillation unit 1 and the second distillation unit 2.

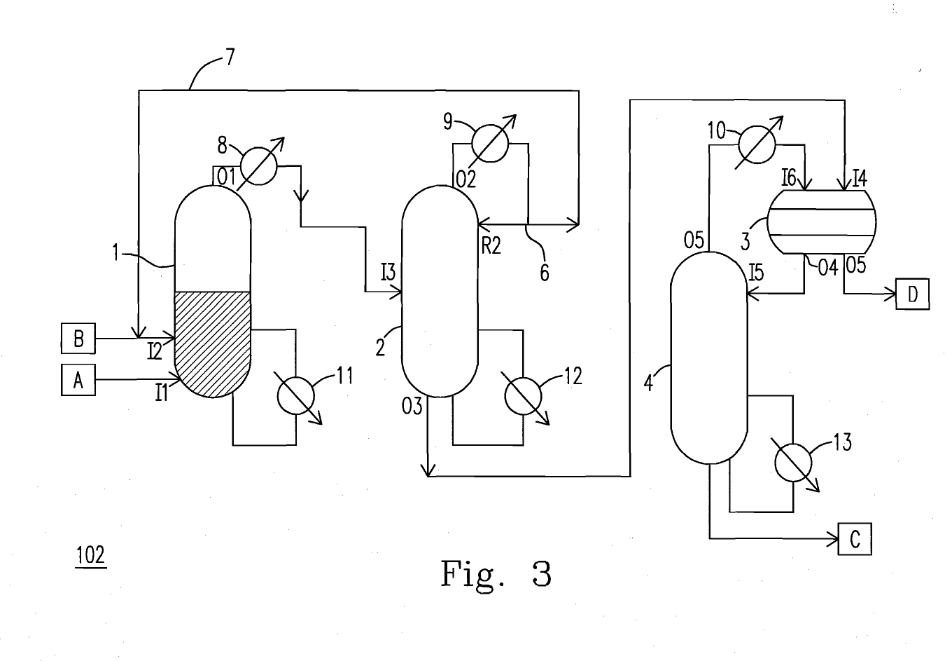

Referring to FIG. 3, it represents a diagram illustrating the system for producing a diester according to a second varied embodiment of FIG. 1. In FIG. 3, the system for producing a diester 102 according to a second varied embodiment is further configured with a second bypass device 6 connected to the second distillation unit 2.

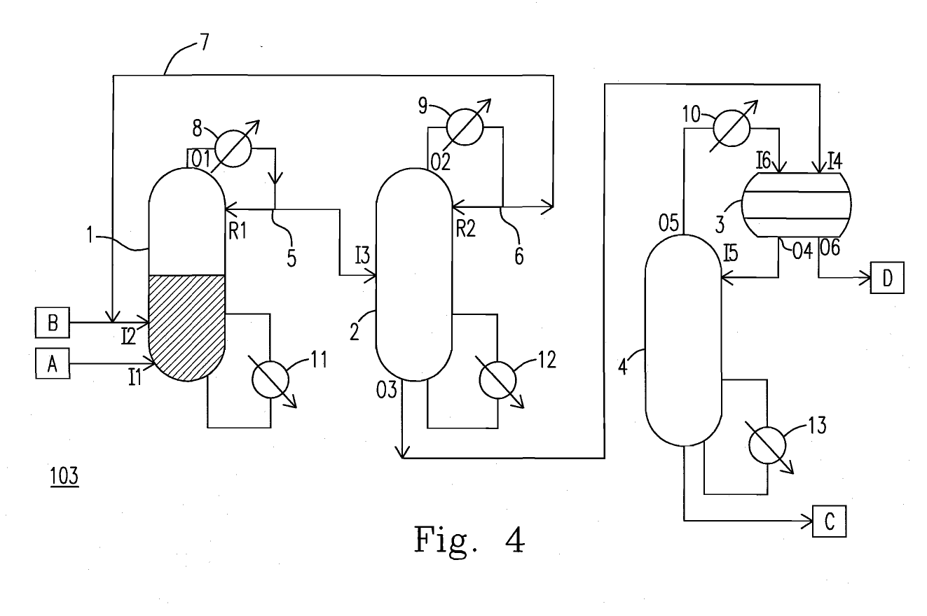

Referring to FIG. 4, it represents a diagram illustrating the system for producing a diester according to a third varied embodiment of FIG. 1. In FIG. 4, the system for producing a diester 103 according to a third varied embodiment is further configured with a first bypass device 5 connected to the first distillation unit 1 and a second bypass device 6 connected to the second distillation unit 2.

Referring to FIG. 1, in a preferred embodiment, the first distillation unit 1 is a reactive distillation column with a rectifying section and a reacting section. The reacting section refers to the section filled with a solid catalyst in the column trays, in which the reacting section is configured in an upper-middle to bottom section or a middle to bottom section. As for the rectifying section, it is configured above the reacting section.

Preferably, the reactants of a dicarboxylic acid (preferably C₄ or higher dicarboxylic acid, and more preferably succinic acid, glutaric acid, adipic acid or the combination thereof) and a alcohol (preferably methanol, ethanol, isopropanol or the combination thereof) are respectively fed from a bottom of the first distillation unit 1 or a first reboiler 11. Specifically, the dicarboxylic acid and the alcohol are respectively fed from a dicarboxylic acid feedstock A through a feed port I1 and an alcohol feedstock B through a feed port I2 into the first distillation unit 1, in which both of the feed ports I1 and I2 are located at the bottom of the first distillation unit 1. In the process, there must be an excess of the alcohol feed in comparison with the dicarbyxylic acid feed, in which the feed ratio of alcohol to the dicarboxylic acid is ranged from about 1.0:1.0 to 1.0:1.0:30.0, so that the above reversible reaction of producing the diester performs forwards.

A catalyst for affecting the reaction is typically divided into homogenous catalyst and heterogenous catalyst. In the reactive distillation column designed according to the invention, heterogenous solid catalyst is mainly applied to increase the reaction. Since the solid catalyst can be filled and configured in any part of the reactive distillation column (e.g. bottom section or top section), the selection of location of the reacting section in the distillation column is flexible without the problems raised in recycling the liquid catalyst. The solid catalyst is normally an ion exchange resin, e.g. industrial Amberlyst 15 (Rohm and Hass), Amberlyst 35 (Rohm and Hass) or Purolite CT179 (Purolite). The filling structure for the catalyst can be constructed by the widely used Katapak-S or Davy Process Technology of placing a fixing device among the column trays (Davy Process Technology).

Since a large quantity of dicarboxylic acid will be remained in the column bottom portion, the catalyst is filled in the bottom trays of the reactive distillation column with preferably 10 to 100 times amount of that in tray column for promoting the reactions.

Preferably, when the procedure of reaction distillation in the first distillation unit is performed, the operating temperature can be manipulated in the range of 60˜225° C. by the first reboiler 11. As described in the above, in the first distillation unit 1, the alcohol is reacted with the carboxylic acid to yield the diester product, monoester product, and water with the remained unreacted dicarboxylic acid and alcohol. In the procedure, the alcohol, the diester and water are turned to be a constant boiling mixture and become a gaseous mixture mainly of the alcohol, the diester and the water. The gaseous mixture moves toward the rectifying section of the first distillation unit 1, and a liquid mainly of heavy content of the high-boiling dicarboxylic acid reactant and monoester is remained in the bottom of the first distillation unit 1.

Preferably, the first gaseous mixture exits from a discharge port O1 on the top of the first distillation unit 1. The first gaseous mixture flows through the first condenser 8, is cooled into a first liquid mixture, and then enters the second distillation unit 2 through a feed port I3.

Referring to FIGS. 2 and 4, the first liquid mixture can be split by a first bypass device 5, e.g. T-type bypass conduit, into a first portion and a second portion before the first liquid mixture enters the second distillation unit 2. Preferably, the first bypass device 5 includes a first end connected to the first distillation unit 1 for receiving the first liquid mixture, a second end connected to the second distillation unit 2 for introducing the first portion of the first liquid mixture into the second distillation unit 2 for separating the first portion to produce the second gaseous mixture and the second liquid mixture, and a third end connected to the first distillation unit 1 for returning a second portion of the first liquid mixture to the first distillation unit 1. The first portion of the first liquid mixture enters the second distillation unit 2 through the feed port I3, and the second portion of the first liquid mixture returns into the first distillation unit 1 through a return port R1. In the process, the temperature in the region between the first bypass device 5 and the return port R1 is operated in the range of from the ambient temperature to 100° C., so that the acid and monoester probably existing in the first liquid mixture can be recycled into the first distillation unit 1 for esterification.

The first liquid mixture enters the second distillation unit, is separated via distillation, and yields a lower boiling second gaseous mixture mainly of the alcohol and a higher boiling second liquid mixture mainly of the diester and the water. In the second distillation unit 2, the temperature is preferably operated in the range of 50˜120° C. Preferably, the second distillation unit 2 may be a stripper (whose configuration is as shown in FIGS. 1 and 2) or a conventional distillation column (whose configuration is as shown in FIGS. 3 and 4). With respect to the stripper, the feed port I3 is located at the column top, so that the first liquid mixture can enter the second distillation unit 2 through the column top thereof. The stripper has a single manipulated variable for controlling a reboiler duty and adjusting the production of the second gaseous mixture, and is adapted to separate the alcohol and the diester respectively with very distinctive boiling points. For example, the diester is the diester of the adipic acid reactant. After the separation, the second gaseous mixture yielded therefrom is recycled to the alcohol feedstock B through the alcohol recycle path 7.

However, if the alcohol and the diester existing in the second distillation unit 2 do not have distinctive boiling points, the constant boiling mixture of the alcohol and the diester may be recycled with the alcohol. For example, the diester is the diester of the glutaric acid reactant. Therefore, the conventional distillation column can be considered when it has a first manipulated variable for controlling a reboiler duty that adjusts production of the second gaseous mixture and a second manipulated variable for controlling a reflux ratio that adjusts a proportion of the diester in the third liquid mixture. The feed port I3 is located at the middle of the conventional distillation column, so that the first liquid mixture can be fed through the middle section of the column.

Referring to FIGS. 3 and 4, the second gaseous mixture mainly of the alcohol exits from the second distillation unit 2 (i.e. conventional distillation column) through a discharge port O2, and is cooled by the second condenser 9 into a third liquid mixture mainly of the alcohol. The third liquid mixture is split by the second bypass device 6, e.g. T type bypass conduit, into a first portion and a second portion. The first portion of the third liquid mixture is recycled to the alcohol feedstock B through the alcohol recycle path 7, and the second portion of the third liquid mixture is returned into the second distillation unit 2 for controlling the proportion of the diester.

Therefore, the unreacted alcohol can exit from the second distillation unit 2 and is recycled to the alcohol feedstock B for reuse and reaction in the first distillation unit. Moreover, depending on the need of the forward esterification in the first distillation unit 1, the alcohol path 7 can be designed for controlling the molar ratio of the fed alcohol and the fed carboxylic acid to exceed the stoichiometric ratio. Preferably, the molar ratio is in the range of from 1.0:1.0 to 1.0:30.0.

The liquid mixture mainly of the diester and the water is substantially a constant boiling product of dual components, and is the heavy content with a relatively higher boiling point. The second liquid mixture exits from the bottom of the second distillation unit 2 through the discharge port O3 thereon, may enter the liquid-liquid extraction unit 3 through the feed port I4.

The liquid-liquid extraction unit 3 can be a decanter, which separating an organic phase from an aqueous phase on the basis of the principle that the organic phase is immiscible with the aqueous phase. Therefore, the liquid-liquid extraction unit 3 separates the second liquid mixture mainly of the diester and the water into an organic-rich mixture mainly of the diester and an aqueous-rich mixture. The organic-rich mixture exits from the liquid-liquid extraction unit 3 through a discharge port O4 and is introduced into the ester recycle unit 4 through a feed port I5 on the top thereof. As for the aqueous-rich mixture, it exits from the liquid-liquid extraction unit 3 through a discharge port O6 and is introduced into a water storage unit D.

The organic-rich mixture mainly of the diester is further purified and separated by e.g. distillation and stripping in the ester recycle unit 4. The ester recycle unit 4 is operated at temperature of preferably 60˜220° C., controlled by the third reboiler 13. Therefore, the separated diester product is a heavy content, and can be collected from the bottom of the ester recycle unit 4 and turned to be the high-purity diester product.

Even though the ester recycle unit 4 may have the water content, the water can be separated and formed to be a gas which exits from the a discharge port O5 and flows through the third condenser 10, and be cooled into an aqueous-rich liquid, returned into the liquid-liquid extraction unit 3 through a feed port I6.

Accordingly, the invention can be applied for manufacturing the high-purity diester, such as dimethyl succinate, dimethyl glutarate, dimethyl adipate or the combination thereof. The complete production procedures can be mainly involved in a reactive distillation column, an alcohol recycle column, a decanter and an ester recycle column. The reactants, methanol and a dicarboxylic acid such as succinic acid, glutaric acid, adipic acid or the combination thereof are fed from bottom trays of the reactive distillation column. The reactive distillation column can be basically divided into a rectifying section and a reacting section, in which the reacting section is filled or partly filled with the solid catalyst. The mixture of the alcohol, the diester, and the water obtained from the top of the reactive distillation column is separated by the alcohol recycle column, and a methanol-rich stream produced from the top of the alcohol recycle column is returned to an alcohol feedstock providing the alcohol and admixed and introduced therewith into the reactive distillation column. A stream having the mixture of the water and the diester from the bottom of the alcohol recycle column is delivered into the decanter, the mixture of the water and the diester is separated by the decanter, and the obtained organic-rich mixture is returned to and stripped in the top of the ester recycle column, so that the high-purity diester product is obtained in the bottom portion of the ester recycle column.

### EXAMPLES

### Example 1

### Production of Dimethyl Glutarate

Example 1 illustrates the reactive distillation system, in which the feeding ratio of methanol to glutaric acid is 6, and the reactive distillation column is operated at the temperature in the range of 70 to 180° C., and the condenser therefor has a controlling temperature of about 65° C. The alcohol recycle column is a conventional distillation column, with the temperature operated in the range of 60 to 110° C. The ester recycle column is operated at the temperature in the range of 90 to 200° C., and the condenser therefor has a controlling temperature of about 90° C. The catalyst is Amberlyst-35. The features of Example 1 are as shown in the following Table 3, in which the concentration (mole fraction) of the dimethyl glutarate product is 0.99000.

TABLE 3

| | System | | |
|---|---|---|---|
| | Dimethyl glutarate (Example 1) | | |
| | Reactive | Alcohol | |
| | distillation | recycle | Ester recycle |
| Item | column | column | column |
| Total theoretical tray | 21 | 22 | 3 |
| number | | | |
| Theoretical stripping trays | — | 15 | — |
| Theoretical reactive trays | 12 | — | — |
| Theoretical rectifying trays | 9 | 7 | — |
| Dicarboxylic acid feed | Bottom trays | — | — |
| trays | | | |
| Alcohol feed trays | Bottom trays | — | — |
| Concentration of | 1 | — | — |
| dicarboxylic acid feed | | | |
| (mole fraction) | | | |
| Concentration of alcohol | 1 | — | — |
| feed (mole fraction) | | | |
| Molar ratio of alcohol feed | 6 | — | — |
| to dicarboxylic acid feed | | | |
| Concentration of diester | — | — | 0.99000 |
| product (mole fraction) | | | |
| Decanter (° C.) | 50 | — | — |

### Example 2

### Production of Dimethyl Adipate

Example 2 illustrates the reactive distillation system, in which the feeding ratio of methanol to adipic acid is 15.6, the reactive distillation column is operated at the temperature in the range of 60 to 190° C., and the condenser therefor has a controlling temperature of about 70° C. The alcohol recycle column is a stripper, with the temperature operated in the range of 60 to 110° C., and the condenser therefor has a controlling temperature of about 65° C. The ester recycle column is operated at the temperature in the range of 90 to 200° C., and the condenser therefor has a controlling temperature of about 93° C. The catalyst is Amerlyst© 15 or Amerlyst© 35. The features of Example 2 are as shown in the following Table 4, in which the concentration (mole fraction) of the dimethyl adipate product is 0.99000.

TABLE 4

| | System | | |
|---|---|---|---|
| | Dimethyl adipate (Example 2) | | |
| | Reactive | Alcohol | |
| | distillation | recycle | Ester recycle |
| Item | column | column | column |
| Total theoretical tray | 2 | 14 | 3 |
| number | | | |
| Theoretical stripping trays | — | — | — |
| Theoretical reactive trays | 13 | — | — |
| Theoretical rectifying trays | 9 | — | — |
| Dicarboxylic acid feed | Bottom trays | — | — |
| trays | | | |
| Alcohol feed trays | Bottom trays | — | — |
| Concentration of | 1 | — | — |
| dicarboxylic acid feed | | | |
| (mole fraction) | | | |
| Concentration of alcohol | 1 | — | — |
| feed (mole fraction) | | | |
| Molar ratio of alcohol feed | 6 | — | — |
| to dicarboxylic acid feed | | | |
| Concentration of diester | — | — | 0.99000 |
| product (mole fraction) | | | |
| Decanter (° C.) | 50 | — | — |

### Example 3

### Production of Dimethyl Glutarate/Dimethyl Adipate

Example 3 illustrates the reactive distillation system, in which the feed ratio of methanol to the mixture of glutaric acid and adipic acid is 20.2, the reactive distillation column is operated at the temperature in the range of 60 to 195° C., and the condenser therefor has a controlling temperature of about 60° C. The alcohol recycle column is a conventional distillation column, with the temperature operated in the range of 60 to 110° C., and the condenser therefor has a controlling temperature of about 65° C. The ester recycle column is operated at the temperature in the range of 90 to 200° C., and the condenser therefor has a controlling temperature of about 98° C. The catalyst is Amerlyst© 15 or Amerlyst© 35. The features of Example 3 are as shown in the following Table 5, in which the concentration (mole fraction) of the produced mixture of dimethyl glutarate and dimethyl adipate is 0.99000.

TABLE 5

| | System | | |
|---|---|---|---|
| | Dimethyl esters of adipic acid and | | |
| | glutaric acid (Example 3) | | |
| | reactive | Alcohol | |
| | distillation | recycle | Ester recycle |
| Item | column | column | column |
| Total theoretical tray | 34 | 21 | 3 |
| number | | | |
| Theoretical stripping trays | — | 15 | — |
| Theoretical reactive trays | 28 | — | — |
| Theoretical rectifying trays | 6 | 6 | — |
| Dicarboxylic acid feed | Bottom trays | — | — |
| trays | | | |
| Alcohol feed trays | Bottom trays | — | — |
| Concentration of | Adipic acid/ | — | — |
| dicarboxylic acid feed | glutaric | | |
| (mole fraction) | acid | | |
| | (0.9/0.1) | | |
| Concentration of alcohol | 1 | — | — |
| feed (mole fraction) | | | |
| Molar ratio of alcohol feed | 20.2 | — | — |
| to dicarboxylic acid feed | | | |
| Concentration of diester | — | — | 0.99000 |
| product (mole fraction) | | | |
| Decanter (° C.) | 50 | — | — |

In summary, the invention can be designed on basis of the thermodynamical properties of different esterification/separation system so as to obtain a high-purity diester product of industrial grade and achieve the advantages of simple use, economy, environmental protection and high efficiency.

Based on the above descriptions, it is understood that the present invention is indeed an industrially applicable, novel and non-obvious one with values in industrial development. While the invention has been described in terms of what are presently considered to be the most practical and preferred embodiments, it is to be understood that the invention should not be limited to the disclosed embodiment. On the contrary, it is intended to cover numerous modifications and variations included within the spirit and scope of the appended claims which are to be accorded with the broadest interpretation so as to encompass all such modifications and variations. Therefore, the above description and illustration should not be taken as limiting the scope of the present invention which is defined by the appended claims.

What is claimed is:

1. A system for producing a diester, comprising:
a first distillation unit reacting a dicarboxylic acid with an alcohol to produce a first gaseous mixture;
a first cooling unit connected to the first distillation unit and cooling the first gaseous mixture into a first liquid mixture;
a second distillation unit connected to the first cooling unit and separating the first liquid mixture into a second gaseous mixture and a second liquid mixture; and
a liquid-liquid extraction unit connected to the second distillation unit and separating the second liquid mixture into an aqueous-rich mixture and an organic-rich mixture from which the diester is obtained.

2. The system as claimed in claim 1 further comprising an ester recycle unit connected to the liquid-liquid extraction unit and separating the organic-rich mixture for obtaining the diester.

3. The system as claimed in claim 2, wherein the ester recycle unit is a third distillation unit.

4. The system as claimed in claim 1, wherein the dicarboxylic acid has a total carbon number of no less than 6.

5. The system as claimed in claim 4, wherein the dicarboxylic acid is one selected from a group consisting of succinic acid, glutaric acid, adipic acid and a combination thereof, and the alcohol is one selected from a group consisting of methanol, ethanol, isopropanol, butanol and a combination thereof.

6. The system as claimed in claim 1, wherein the alcohol and the dicarboxylic acid have a molar ratio ranged from 1.0:1.0 to 1.0:30.0.

7. The system as claimed in claim 1, wherein the first gaseous mixture is a mixture of the alcohol, the diester and water, the second gaseous mixture is an alcohol-rich mixture, and the second liquid mixture is a mixture of the diester and the water.

8. The system as claimed in claim 1, wherein the first distillation unit comprising:
a plurality of reactive trays configured in a bottom to middle section of the first distillation unit;
a plurality of rectifying trays configured in a middle to top section of the first distillation unit; and
a solid catalyst filled in a middle to bottom section of the first distillation unit.

9. The system as claimed in claim 1 further comprising:
a first bypass device comprising:
a first end connected to the first distillation unit for receiving the first liquid mixture;
a second end connected to the second distillation unit for introducing a first portion of the first liquid mixture into the second distillation unit for separating the first portion to produce the second gaseous mixture and the second liquid mixture; and
a third end connected to the first distillation unit for returning a second portion of the first liquid mixture to the first distillation unit.

10. The system as claimed in claim 1 further comprising:
an alcohol feedstock connected to the first distillation unit and providing the alcohol;
a second cooling unit connected to the second distillation unit and cooling the second gaseous mixture into a third liquid mixture; and
an alcohol recycle path configured between the second distillation unit and the alcohol feedstock and recycling the third liquid mixture to the alcohol feedstock.

11. The system as claimed in claim 10 further comprising a second bypass device connected to the second distillation unit for splitting the third liquid mixture into a first portion to be introduced to the alcohol feedstock and a second portion to be returned to the second distillation unit.

12. The system as claimed in claim 1, wherein the second distillation unit has a single manipulated variable for controlling a reboiler duty and adjusting production of the second gaseous mixture.

13. The system as claimed in claim 12, wherein the second distillation unit is a stripper.

14. The system as claimed in claim 12, wherein the dicarboxylic acid is adipic acid.

15. The system as claimed in claim 1, wherein the second distillation unit has a first manipulated variable for controlling a reboiler duty and adjusting production of the second gaseous mixture and a second manipulated variable for controlling a reflux ratio and adjusting a proportion of the diester in the third liquid mixture.

16. The system as claimed in claim 15, wherein the second distillation unit is a typical distillation column.

17. The system as claimed in claim 15, wherein the dicarboxylic acid is glutaric acid.

18. A method for producing a diester, comprising the steps of:
admixing a dicarboxylic acid and an alcohol to produce a first gaseous mixture via distillation;
cooling the first gaseous mixture to form a first liquid mixture;
separating the first liquid mixture by the distillation to produce a second gaseous mixture and a second liquid mixture;
separating the second liquid mixture into an organic-rich mixture and an aqueous-rich mixture by liquid-liquid extraction; and
separating the organic-rich mixture to produce the diester.

19. The method as claimed in claim 18 further comprising a step of cooling the second gaseous mixture to form a third liquid mixture and introducing the third liquid mixture into the alcohol.

20. The method as claimed in claim 18, wherein the organic-rich mixture is separated by distillation to produce the diester.
